(19) United States
(12) Patent Application Publication
(10) Pub. No.: US 2004/0137082 A1
(43) Pub. Date: Jul. 15, 2004

(54) Compounds and methods for reducing myostatin concentration

(76) Inventor: Zakir Ramazanov, Warwick, NY (US)

Correspondence Address: SULLIVAN LAW GROUP, 1850 NORTH CENTRAL AVENUE, SUITE 1140, PHOENIX, AZ, 85004, US

(21) Appl. No.: 10/340,210

(22) Filed: Jan. 9, 2003

(51) Int. Cl. A61K 35/78; A61K 31/715

(52) U.S. Cl. 424/725; 514/61

(57) ABSTRACT

Compounds and methods are provided for reducing myostatin concentration. The compounds include an extract of Cystoseira canariensis having at least one sulfated polysaccharide. Each of the at least one sulfated polysaccharides preferably has fewer than nine saccharide units. The compounds also may include fluorotannins, fluoroglucinols or both.

### BACKGROUND OF THE INVENTION

1. Field of the Invention

The present invention relates to compounds and methods for affecting myostatin concentration, for example, as a means to impact muscle tissue development, and to compounds and methods for facilitating maintenance or increases in muscle mass by addressing myostatin concentration.

2. Description of the Related Art

The ability to regulate muscle development is advantageous in a number of respects. The ability to maintain or increase muscle mass is useful, for example, not only in maintaining muscle tone and development but also to offset normal loss of muscle mass as one ages. It also can be used to treat various diseases and ailments that involve muscle tissue deterioration as a symptom or manifestation.

It has been found that the protein myostatin functions as a negative regulator of skeletal muscle mass, apparently without side effects on other muscles. Muscles can grow uninhibited when myostatin function or genes are inhibited. This discovery was made based on observations of certain animal species that naturally exhibited pronounced muscle mass relative to similar species, a phenomenon commonly called “double muscling” in the literature. The most notable example is Belgian Blue cattle and Peidmontese cattle. A. McPherson and S. Lee, “Double Muscling In Cattle Due To Mutations In The Myostatin Gene,” Proc. Nat'l Acad. Sci. USA, Vol. 94, pp. 12457-12461, November 1997. The significantly increased muscle mass observed in these cattle was found to be due to a lack of functional myostatin protein. This aspect of myostatin apparently was first described by McPherron et al. in 1997. McPherron, A. C., Lawler, A. M., Lee, S. J., “Regulation of Skeletal Muscle Mass In Mice By A New TGF.Beta.Superfamily Member,” Nature, 387-83-90 (1997). Myostatin-null mice show a dramatic and widespread increase in skeletal muscle mass due to an increase in number of muscle fibers (hypertrophy). One such mouse exhibited average muscle weight gain of 261 percent. Similar results also are reported with respect to Belgian Blue cattle.

Myostatin belongs to a family of molecules known as transforming growth factors—beta (“TGF-b”). It also is called “growth and differentiation factor—8” (“GDF-8”). Growth factors (“GF”) are normally effective in very low concentrations and have high affinity for their corresponding receptors on target cells. A group of GFs is the transforming growth factor beta (“TGF-b”) superfamily of which there are several subtypes based on their related structures. A common feature of TGF-b members is that they are secreted by cells in an inactive complex form. GDF-8 is one of three structures that specifically regulate growth and differentiation.

Myostatin genes encode secreted factors that are important for regulating embryonic development and tissue homeostasis in adults. Myostatin protein purified from mammalian cells comprises a non-covalently held complex of an N-terminal propeptide and a disulfide-linked dimer of C-terminal fragments. Information on myostatin structure is provided, for example, in U.S. Pat. No. 5,827,733, issued to Lee et al. on Oct. 27, 1998. The structure of myostatin reportedly is very common among various species. The myostatin coding sequence of Belgian Blue cattle has an 11-nucleotide deletion, which ultimately results in expression of a truncated protein product. Peidmontese cattle also express a nonfunctional myostatin protein due to a missense mutation in the gene sequence. Kambadur, R., Sharma, M., Smith, T. P. L., Bass, J. J., “Mutations In Myostatin (GDF8) In Double-Muscled Belgian Blue and Piedmontese Cattle,” Genome Res 7,910-915 (1997); Grobet, L., Martin, L. J. R., Poncelet, D., Pirottin, D., Brouwers, B., Riquet, J., Schoeberlein, A., Dunner, S., Menissier, F., Massabanda, J., Fries, R., Hanset, R., Georges, M., “A Deletion In The Bovine Myostatin Gene Causes The Double-Muscled Phenotype In Cattle,” Nature (London) Genetics 17, 71-74 (1997); McPherron et al., 1997.

Myostatin null mutants exhibit both muscle hypertrophy and hyperplasia. McPherron et al. 1997. Muscle represents the balance between muscle cell replication and protein synthesis and muscle protein breakdown and cell death. It is believed that myostatin inhibits muscle growth by affecting one or more of these processes. Myostatin is produced as a precursor protein, which contains a propeptide and an active ligand. According to McPherron et al., 1977, proteolysis of the precursor protein releases mature myostatin. Both precursor protein and active myostatin form disulfide-linked dimers. McPherron et al. 1977. It has been demonstrated that myostatin circulates in serum as part of a latent complex. Zimmers et al., 2002.

In view of these findings, a number of approaches and methods have been proposed or considered to limit or reduce the concentration of myostatin in vivo. Theoretically, such approaches may include reducing the production of myostatin, for example, by suppressing the myostatin gene and/or its expression of myostatin. Another such approach involves attempting to reduce the concentration of myostatin. In principle, this may be accomplished by changing the structure of the molecule, by binding its reaction sites, or otherwise mitigating its function. In U.S. Pat. No. 6,369,201, for example, methods are reported for modifying the myostatin by converting it into a “myostatin immunogen,” whereby the immune system of the subject will deactivate and/or remove it through immunological response.

Few if any methods or compounds for effectively binding or chemically altering myostatin, however, are known or reported in the literature, especially wherein such compounds or methods are amenable to in vivo application.

### OBJECTS OF THE INVENTION

Accordingly, an object of the present invention is to provide compounds and/or methods that may be used to reduce myostatin concentration.

Another object of the invention is to provide compounds and/or methods that are capable of facilitating muscle growth and development.

Another object of the invention is to provide compounds and/or methods that are capable of reducing the effects of myostatin in impairing muscle development.

Another object according to certain aspects of the invention is to provide a method for making a plant or material that may be used in reducing myostatin concentration.

Additional objects and advantages of the invention will be set forth in the description which follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve the foregoing objects, and in accordance with the purposes of the invention as embodied and broadly described in this document, a compound is provided for reducing myostatin concentration. The compound comprises an extract of Cystoseira canariensis comprising at least one sulfated polysaccharide. Naturally occurring Cystoseira canariensis is a marine vegetable found in ocean environments, most notably in the Atlantic Ocean. Beds of Cystoseira canariensis are located, for example, in the Atlantic Ocean in the general vicinity of the Canary Islands. They are indigenous to the Canary Archipelago (Spain). Preferred sulfated polysaccharides comprise those having fewer than nine saccharide units. Particularly preferred sulfated polysaccharides comprise mono-sulfosaccharides, di-sulfosaccharides, tetra-sulfosaccharides, octa-sulfosaccharides, and combinations of these. The compound also optionally but preferably comprises at least one fluorotannin (phlorotanning), at least one fluoroglucinol (phloroglucinol), or both.

In accordance with another aspect of the invention, a method is provided for reducing myostatin concentration. The method comprises applying to myostatin at least one sulfated polysaccharide. Those sulfated polysaccharides noted above are preferred, and the compound comprising the sulfated polysaccharide may comprise at least one fluorotannin or at least one fluoroglucinol, or both, also as noted above.

In accordance with another aspect of the invention, a method is provided for reducing myostatin concentration in a subject. The method comprises administering to the subject at least one sulfated polysaccharide in an amount effective to reduce the myostatin concentration in the subject. In this method as well, preferred sulfated polysaccharides include those described above, and the compound may comprise at least one fluorotannin, or at least one fluoroglucinol, or both.

In accordance with another aspect of the invention, a method is provided for producing Cystoseira canariensis. The method comprises obtaining a Cystoseira canariensis starting material, and growing the Cystoseira canariensis starting material in a vessel containing a liquid growth medium comprising water to produce the Cystoseira canariensis. The growing comprises circulating a portion of the liquid growth medium within the vessel adjacent to the Cystoseira canariensis starting material.

The Cystoseira canariensis starting material preferably comprises naturally occurring Cystoseira canariensis. It is also preferred that the Cystoseira canariensis starting material comprises a plurality of different species of naturally-occurring Cystoseira canariensis. The Cystoseira canariensis starting material preferably comprises the Cystoseira canariensis created by the method according to this aspect of the invention. Cystoseira canariensis grown according to the method described herein, for example, may be used as a starting material or feed material to create new batches of Cystoseira canariensis using the method.

The liquid growth medium according to this method may and preferably does comprise seawater. The amount of the liquid growth medium used, or at least the minimum amount, may be specified in terms of a “charge ratio,” which is defined for present purposes to be the starting Cystoseira canariensis mass in kilograms (“kg”) relative to the liquid growth medium volume in liters (“l”). The presently preferred charge ratio is between about 1:8 and about 1:20, and more preferably between about 1:8 and about 1:16. In the preferred examples presented below, the charge ratio is about 1:16.

In the presently preferred implementation of the method, the liquid growth medium is filtered, e.g., to remove or substantially remove solids, algae, microorganisms, and the like. It also is preferred that the liquid growth medium comprise at least one sulfate. Presently preferred sulfates comprise an inorganic sulfate, such as magnesium sulfate. The amount of sulfate enrichment will depend upon the specific application. In the preferred implementation, the at least one sulfate may be quantified in terms of the amount of elemental sulfate that is in the liquid growth medium. It is presently preferred that the amount of the at least one sulfate be such that the amount of elemental sulfate has a concentration of up to about 1,000 mg per liter of the liquid growth medium. The lower end of this range, however, is preferred in many applications. An elemental sulfate concentration of about 100 mg or less, and more preferably about 2 mg per liter of the liquid growth medium is particularly preferred.

It is also preferred in this method that the liquid growth medium be moved or circulated relative to the Cystoseira canariensis starting material and the Cystoseira canariensis as the growth occurs, or similarly that there be movement between the Cystoseira canariensis and the liquid growth medium. This movement may be quantified using flow rates and/or flow rate concepts, as will be described in greater detail below.

One such approach involves the amount of flow of the liquid growth medium that occurs during the time period required for the Cystoseira canariensis to double its mass. The Cystoseira canariensis, for example, has a starting Cystoseira canariensis mass, and the Cystoseira canariensis (starting material plus plant growth) has a Cystoseira canariensis mass that changes as the growing takes place. The “mass doubling growth period” is defined herein as the time period required for the Cystoseira canariensis mass to become twice the starting Cystoseira canariensis mass. Where the growth process is carried out in a batch environment with a fixed or relatively fixed volume of the liquid growth medium, the flow rate of the liquid growth medium may be expressed in terms of the volumetric flow rate of liquid growth medium that flows during a specified time period, such as the mass doubling growth period. In the presently preferred implementation of the method according to this aspect of the invention, the circulating of the liquid growth medium comprises circulating the liquid growth medium with a flow rate of at least the liquid growth medium volume per each of the mass doubling growth periods.

It is also preferred that the liquid growth medium be aerated. This may comprise directing air bubbles toward the Cystoseira canariensis starting material, e.g., by providing an air bubble stream so that the air bubbles move past and contact the Cystoseira canariensis under their own buoyancy. One also may entrain the Cystoseira canariensis in a gas stream, such as air, within the liquid growth medium.

The presently preferred temperature for the liquid growth medium is about 10° C. to about 25° C., and more preferably about 18° C.

It is also preferred to expose the Cystoseira canariensis to a growth light level that is lower than an ambient atmospheric light levels outdoors. Ambient atmospheric light levels are generally about 2,000 μmol/m²-sec. The presently preferred light level for growth according to this method is between about 300 μmol/m²-sec and 1,500 μmol/m²-sec., and more preferably about 600 μmol/m²-sec.

The Cystoseira canariensis produced by the method in its various aspects as summarized herein above comprise yet another aspect of the invention.

In accordance with another aspect of the invention, a method is provided for processing Cystoseira canariensis. The method comprises contacting the Cystoseira canariensis with a solvent to extract a fraction comprising at least one sulfated polysaccharide. The at least one sulfated polysaccharide may comprise those identified herein above. The fraction also may comprise at least one fluorotannin. Additionally or alternatively, it may comprise at least one fluoroglucinol.

The method according to this aspect of the invention preferably but optionally comprises pre-treating the Cystoseira canariensis prior to contacting it with the solvent. The pre-treatment preferably comprises washing the Cystoseira canariensis, preferably with water and more preferably with fresh water. The pre-treatment also may comprise dehydrating the Cystoseira canariensis, freeze drying it, and/or granulating it, preferably prior to the solvent contacting.

Preferred solvents for contacting with the Cystoseira canariensis according to this method comprise water and/or a low-molecular weight alcohol, preferably ethanol, for example, as described in greater detail herein below. The contacting of the Cystoseira canariensis with the solvent according to this aspect of the invention preferably comprises a multi-stage extraction. The presently preferred implementation comprises a three-stage extraction process, also as described in more detail herein below. Fractions produced by the method according to this aspect of the invention, in their various forms, that are effective in reducing myostatin concentration comprise yet another aspect of the invention.

In accordance with still another aspect of the invention, a method is provided for reducing myostatin concentration, wherein the method comprises applying one or more of the fractions, and most preferably the end fraction (“Fraction C”), produced by the above-described method, in its various forms, to the myostatin.

In accordance with yet another aspect of the invention, a method is provided for reducing myostatin concentration of a subject, wherein the method comprises administering to the subject the end fraction produced by the above-described method, in its various forms, in an amount effective to reduce the myostatin concentration in the subject.

### BRIEF DESCRIPTION OF THE DRAWINGS

The accompanying drawings, which are incorporated in and constitute a part of the specification, illustrate a presently preferred implementation of methods of the invention and, together with the general description given above and the detailed description of the preferred versions of the methods given below, serve to explain the principles of the invention. Of the drawings:

FIG. 1 shows a bioreactor used in accordance with a presently preferred implementation of the inventive methods according to one aspect;

FIG. 2 shows an apparatus used to analyze a sample of the Fraction C according to a presently preferred embodiment of the invention, and made according to a presently preferred implementation of a method according to the invention.

FIG. 3 shows the results of a Western blot immuno-electrophoresis analysis on this Fraction C; and

FIG. 4 shows the results a Western blot immuno-electrophoresis analysis on a sample comprising Fraction C and incubated muscle tissue.

### DETAILED DESCRIPTION OF THE

### PREFERRED EMBODIMENTS AND METHODS

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative compounds, devices and methods, and illustrative examples shown and described in this section in connection with the preferred embodiments and methods. The invention according to its various aspects is particularly pointed out and distinctly claimed in the attached claims read in view of this specification, and appropriate equivalents.

In accordance with a first aspect of the invention, a compound is provided for reducing myostatin concentration. The compound comprises an extract of Cystoseira canariensis comprising at least one sulfated polysaccharide. Preferred sulfated polysaccharides include those having fewer than nine saccharide units. Particularly preferred sulfated polysaccharides comprise mono-sulfosaccharides, di-sulfosaccharides, tetra-sulfosaccharides, octa-sulfosaccharides, or combinations of these. The compound also optionally may comprise at least one fluorotannin. Additionally or alternatively, it may comprise at least one fluoroglucinol.

In accordance with another aspect of the invention, a method is provided for reducing myostatin concentration. The method comprises applying to myostatin at least one sulfated polysaccharide. The at least one sulfated polysaccharide preferably comprises the compound or compounds described herein above, and thus comprises an extract of Cystoseira canariensis. Presently preferred sulfated polysaccharides include those identified herein above, and preferably have fewer than nine saccharide units. The method also may comprise the application of at least one fluorotannin, e.g., as a component of the compound comprising the at least one sulfated polysaccharides. Additionally or alternatively, it may comprise at least one fluoroglucinol. The method may be carried out in vivo or in vitro.

In accordance with a related aspect of the invention, a method is provided for reducing myostatin concentration in a subject. The method comprises administering to the subject at least one sulfated polysaccharide in an amount effective to reduce the myostatin concentration in the subject. This method also may be implemented by administering the compound or compounds described herein above, although the method is not necessarily limited only to the compounds as specifically described there. Presently preferred sulfated polysaccharides include those identified above, and preferably have fewer than nine saccharide units. The method also may comprise administration of at least one fluorotannin. It also may comprise administration of at least one fluoroglucinol. The presently preferred compound and the presently preferred method for reducing myostatin concentration using the preferred compound are referred to collectively herein as the “preferred implementation.”

Presently preferred compounds and presently preferred implementations of the method for reducing myostatin concentration involve using an extract from the marine vegetable Cystoseira canariensis that comprises the at least one sulfated polysaccharide. The Cystoseira canariensis used as a raw material or input to make the preferred compound may be a naturally occurring variety obtained by harvesting it from the ocean floor where it grows, or a cultivated or synthetic variety, for example, obtained as described herein below. Cystoseira canariensis is indigenous to the region of the Canary Islands, more specifically the Canary Archipelago, and that source is preferred.

Rather than using a single variety or species of plant, the effectiveness of compounds and methods according to the various aspects of the invention may be improved by using a variety of species of Cystoseira canariensis. These species may be selected according to such criteria as their speed of growth, growth conditions, source location, durability, etc. Using multiple species or varieties can facilitate plant growth and result in yields that are more hearty. In the preferred embodiments and implementations, about 15 different species of Cystoseira canariensis were used as a source material for obtaining the desired cultivation.

Supplies of the naturally-occurring marine vegetable Cystoseira canariensis have been relatively limited. There has been a need, therefore, for alternative sources of it. Accordingly, in accordance with another aspect of the invention, a method is provided for producing Cystoseira canariensis. This method comprises obtaining a Cystoseira canariensis starting material and growing the Cystoseira canariensis starting material in a vessel containing a liquid growth medium comprising water to produce the Cystoseira canariensis.

The Cystoseira canariensis starting material may comprise the naturally occurring vegetable, artificially grown versions of it, or other sources having substantially the same composition as the naturally-occurring vegetable or capable of yielding the substantially the same fractions as are described herein below. Artificially grown versions of the Cystoseira canariensis may be obtained using the method according to this aspect of the invention. As noted above, the preferred implementation of this method involves using multiple sources and/or species of Cystoseira canariensis as the starting material, preferably including a plurality of different species of naturally-occurring Cystoseira canariensis, and preferably but optionally including Cystoseira canariensis created by one or more of the methods described herein.

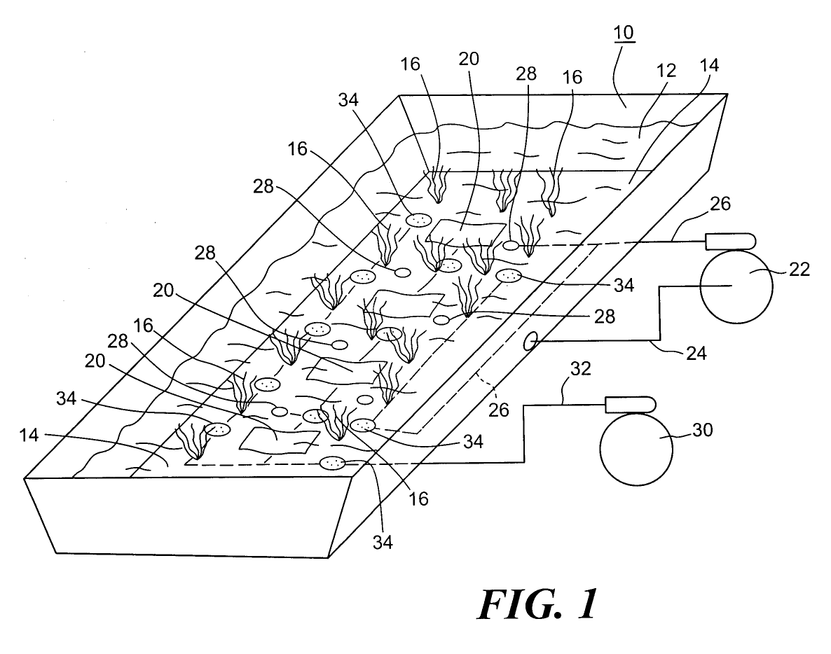

To illustrate this aspect of the invention, a presently preferred but merely illustrative implementation of the method according to this aspect of the invention may be carried out in the apparatus shown in FIG. 1. In this implementation, a commercial bioreactor vessel 10 contains a liquid growth medium 12 in the form of filtered seawater or its substantial equivalent. A sediment 14 comprising that found on the ocean floor in vicinities where Cystoseira canariensis grows naturally, or its substantial equivalent, is disposed in the bottom of vessel 10. A plurality of small or young plants of Cystoseira canariensis, referred to herein as “saplings” 16, are disposed in the sediment 14 within the liquid growth medium 12.

It is preferable that the vessel contain at least a minimum amount of liquid growth medium for a given amount of Cystoseira canariensis starting material or total Cystoseira canariensis contained within the vessel. The amount of liquid growth medium used in a given instance will depend on the specific application. In general, the amount of liquid growth medium should be sufficient to adequately provide nourishment for the saplings and to adequately remove or dilute metabolic wastes. In the presently preferred version of the method, a batch-type processing approach is used, and the concept of the “charge ratio” is useful. As noted above, the Cystoseira canariensis starting material has a starting Cystoseira canariensis mass which, for present purposes, is assumed to be measured in kilograms (kg). The liquid growth medium contained within the vessel has a liquid growth medium volume that is assumed here to be measured in liters (l). The charge ratio is defined as the starting Cystoseira canariensis mass relative to the liquid growth medium volume. Using this definition, preferred charge ratios are between about 1:8 and about 1:20, and more preferably between about 1:8 and about 1:16. In the presently preferred implementation of this method, the charge ratio is about 1:16. In the preferred implementation of the method, the seawater is filtered in the sense that solids, algae and microorganisms have been removed to the extent possible or practical and reasonable under the circumstances.

It is preferred that the liquid growth medium be filtered. This filtering may comprise filtering to remove solids, algae, microorganisms, and/or the like, and preferably all of these.

The liquid growth medium preferably is sulfate enriched, and thus comprises at least one sulfate. Preferred sulfates are those that provide a good source of elemental sulfate in solution in the liquid growth medium. It is preferred, for example, that the at least one sulfate comprises an inorganic sulfate, such as magnesium sulfate (e.g., MgSO₄.7H₂O). The extent of sulfate enrichment will depend upon the specific application. Preferably, the sulfate enrichment is such that the elemental sulfate has a concentration of up to about 1,000 milligrams (mg) per liter of the liquid growth medium. In the presently preferred implementation of the method, the sulfate enrichment is such that the elemental sulfate has a concentration of about 2 mg per liter of the liquid growth medium. The manner in which the sulfate enrichment occurs is not necessarily limiting. Sulfate or sulfate-containing media may be gradually introduced using a by-pass flow or reflow of the liquid growth medium. Known automatic control techniques and/or equipment may be employed to regulate the concentration of sulfate in the liquid growth medium. It is also possible, for example, to place a store of the sulfate source in the liquid growth medium and to time release it. With reference to FIG. 1, for example, this may be accomplished by placing plastic bags 20 of inorganic sulfate into the bottom of vessel 10, wherein apertures have been placed in the bags to allow for the timed release of the sulfate into the liquid growth medium.

The growth of these saplings according the presently preferred implementation of the method also comprises circulating or otherwise moving a portion of the liquid growth medium within the vessel adjacent to the Cystoseira canariensis starting material. Although not wishing to be limited by any particular theory of operation, this movement or circulation can be used to facilitate plant health and growth, for example, by providing nutrients to the saplings, and by removing or diluting metabolic wastes that otherwise might accumulate at the saplings.

The extent of the circulation will depend upon the specific application, but generally should be sufficient, as noted, to provide adequate nourishment to the saplings, and/or to adequately remove or dilute metabolic wastes at the saplings. Quantitative guidance may be provided using the concept of a flow rate. The Cystoseira canariensis starting material has a starting Cystoseira canariensis mass, which of course can be directly measured. The total amount of Cystoseira canariensis in the vessel at a given time has what will be referred to herein as a “Cystoseira canariensis mass,” which also can be measured. The Cystoseira canariensis mass changes as the growing of the Cystoseira canariensis takes place in the vessel. The “mass doubling growth period” is defined herein as the time period required for the total Cystoseira canariensis mass to become twice the starting Cystoseira canariensis mass. The liquid growth medium flow rate can be defined as the amount of liquid growth medium (by mass or volume) that is displaced during the mass doubling growth period.

It is preferred that the liquid growth medium is circulated such that the liquid growth medium flow rate is at least equal to the liquid growth medium volume or mass per each of the mass doubling growth periods, e.g., the flow rate is such that at least the volume or mass of the initial charge of liquid growth medium is displaced during the mass doubling growth period. Greater flow rates may be used, but excessive flow rates that may cause damage to the saplings should be avoided. In the presently preferred implementation of this method, a flow rate equal to the volume of liquid growth medium in vessel 10 per each mass doubling growth period is used. This flow is implemented using a pump 22 with an input line 24 disposed in the liquid growth medium within vessel 10 and output lines 26 coupled to a plurality of outlets 28 in the bottom and/or sides of vessel 10. Only representative ones of the lines are shown in FIG. 1 to simplify it. Flow is regulated by adjusting the output of pump 22.

It is also preferred that the liquid growth medium is aerated. Although again not wishing to be limited to any particular theory of operation, aeration can provide another means to provide nutrients to the saplings, and to remove or dilute metabolic wastes and other potentially harmful substances. The aeration may, for example, comprise exposing the Cystoseira canariensis starting material to air bubbles in the liquid growth medium. One approach for growing of the Cystoseira canariensis starting material comprises entraining the Cystoseira canariensis in a gas stream within the liquid growth medium, wherein the gas stream preferably comprises air. Other techniques also may be used to expose the Cystoseira canariensis vegetables to this air flow. In the preferred version of this method, this is implemented using an aerating pump 30 with output lines 32 coupled to a plurality of outlets 34 in the bottom of vessel 10 adjacent to the Cystoseira canariensis saplings. The air flow rate is regulated by adjusting the output of pump 30.

The temperature of the liquid growth medium preferably is maintained during Cystoseira canariensis growth to a range of about 10° C. to about 25° C. The preferred temperature for the presently preferred implementation of the method is a temperature of about 18° C. Given that the Cystoseira canariensis is immersed in the liquid growth medium, its temperature also generally will be substantially the same as that of the growth medium and thus will lie generally within these ranges.

It is also preferred that the Cystoseira canariensis during its growth is exposed to a growth light level that is lower than an ambient atmospheric light level, preferably from about one fourth to about three fourths that of ambient light, and more preferably about half or less. Ambient atmospheric light levels on land are generally about 2,000 μmol/m²-sec. In preferred versions of this method, the growth light level preferably is between about 300 μmol/m²-sec and 1,500 μmol/m²-sec., and more preferably is about 600 μmol/m²-sec. It is preferred that any ultraviolet component of the light used in the method be substantially reduced relative to ambient levels.

This method in its various preferred forms may be used to yield Cystoseira canariensis that can used as in input into methods according to other aspects of the invention to produced desired compounds. The Cystoseira canariensis itself as produced according to these methods comprises yet another aspect of the invention.

In accordance with another aspect of the invention, a method is provided for processing Cystoseira canariensis. The method according to this aspect of the invention comprises contacting the Cystoseira canariensis with a solvent to extract a fraction comprising at least one sulfated polysaccharide. The at least one sulfated polysaccharide may comprise those identified herein above, e.g., one or more sulfated polysaccharides, preferably having fewer than nine saccharide units. The fraction also may comprise at least one fluorotannin, at least one fluoroglucinol, or a combination of these.

The method preferably comprises pre-treating the Cystoseira canariensis prior to contacting the Cystoseira canariensis with the solvent. This pre-treatment may and preferably does comprise washing the Cystoseira canariensis, preferably with water and more preferably with fresh water or a liquid or fluid consisting essentially of fresh water.

The pre-treatment of the Cystoseira canariensis also may and preferably does comprise dehydrating it. This may be done by exposing it to warm, dry air. This pre-treatment also may and preferably does comprise freeze drying of the Cystoseira canariensis.

The pre-treatment also preferably comprises granulating the Cystoseira canariensis to obtain Cystoseira canariensis particles. This preferably comprises granulating the Cystoseira canariensis so that it is converted to particles having a maximum particle size of about 1 centimeter (cm), and more preferably of no more than about 5 to 10 millimeters (mm). The particle size distribution preferably is about 3 mm to about 1 cm, more preferably about 3 to about 10 mm, and even more preferably about 5 mm to about 10 mm. Although once again not wishing to be limited to any particular theory, these particle size and distribution ranges provide for good surface contacting of the solvent with the Cystoseira canariensis particles, while avoiding limitations that may be introduced by unduly small particle sizes, e.g., such as “muddy” consistency and undue difficulty in subsequent separation.

The solvent used for the contacting with the Cystoseira canariensis can vary depending on the specific application. Presently preferred solvents according to this aspect of the invention comprise water, a low molecular weight alcohol, or combinations of these. Preferred low molecular weight alcohols comprise ethanol. In preferred versions of the method, the low molecular weight alcohol comprises at least about 80 percent by volume of the solvent, at least initially. The solvent, for example, may comprise at least about 20 percent by volume of the water and at least about 80 percent by volume of the low molecular weight alcohol, preferably ethanol, again, at least initially.

In the preferred version of this method, the contacting of the Cystoseira canariensis with the solvent comprises a multi-stage extraction. The specific number of stages, the specific procedures, parameters, etc. for a given stage, and other processing variables may vary depending on a number of factors. In this illustrative yet preferred implementation of the method, a three-stage extraction is employed. The solvent as generally described above may take different forms in the various extraction stages. The solvent used in the first stage accordingly is referred to herein as the “first solvent,” the solvent used in the second stage is referred to as the “second solvent,” and so on.

In the first extraction stage, a first solvent is used to contact the Cystoseira canariensis and to thereby obtain a “first fraction.” The first solvent preferably comprises about 10 to about 30 percent, and more preferably about 20 percent, by volume of water, and about 70 to 90 percent, more preferably about 80 percent, by volume of low molecular weight alcohol, preferably ethanol. The temperature preferably is about 25° C. to about 75° C., and more preferably to about 45° C. The temperature in this instance, and in each of the extraction stages, normally should not exceed about 90° C., e.g., where there is a risk that the alcohol will begin to distill. The first extraction stage preferably is carried out for about 2 to 6 hours, more preferably 2 to 4 hours, and even more preferably about 3 hours. It is preferred that the bath be agitated, e.g., using known mixing techniques, preferably continuously, during this stage. After having contacted the first solvent and the Cystoseira canariensis for the desired time period, the first solvent is distilled to remove the ethanol, e.g., using known separation techniques such as distillation. The liquid fraction remaining, either in its aqueous solution form or after removal of water, is referred to herein the “first fraction.”

### EXAMPLE 1

As a specific example of this preferred method, one kilogram (“kg”) of freeze-dried Cystoseira canariensis marine vegetable obtained as described above was thalli milled to obtain a granulated form with particle sizes of not more than 5 mm. The granulated vegetable was placed into 10-liter glass extraction vessels and extracted with 5 liters of a first solvent consisting essentially of a water and ethanol mixture (20:80 percent by volume, respectively) for 6 hours with intensive agitation at 45-75° C. The liquid extract obtained from this process was distilled under reduced temperature to recover the ethanol. This resulted in a substantially alcohol-free extract. Spray drying is optional. Where spray drying is used, the aqueous liquid extract may be spray dried to remove part or substantially all of the water, thus yielding a solid or a solid containing product. In this particular example, spray drying was implemented using a tower in which the temperature at the top was about 180° C., and at bottom was about 80° C. It yielded a substantially solid powder (about 5 to 10% moisture). The fraction thereby obtained, which again may constitute or include a solid phase, is designated herein as the “first fraction” or “Fraction A.” In this illustrative example it constituted a yield of about 10 to about 12% of large cell-wall fibers and cell walls.

In the second extraction stage, the first fraction is contacted with a second solvent to obtain a second fraction. The second solvent used in this second stage preferably comprises about 30 to about 60 percent (more preferably about 40 percent) by volume of water and about 40 to about 70 percent (more preferably about 60 percent) by volume of a low molecular weight alcohol, preferably ethanol. The temperature preferably is maintained within the same ranges as those described above for the first phase. Agitation also is preferred. The second extraction stage preferably comprises contacting the first fraction with the second solvent for about 2 to about 6 hours, and more preferably about 3 hours to about 5 hours while agitating, preferably continuously. At the completion of this second extraction stage, the resultant liquid preferably is separated, e.g., using known techniques, to remove the ethanol and obtain the second fraction. This second fraction typically will comprise a more concentrated form of the first fraction, comprising pigments, sterols, cell-wall fibers, and mono-sulfated saccharides. It may comprise an aqueous solution or suspension, or it may be in solid phase or substantially in solid phase.

### EXAMPLE 2

As an example of this second stage extraction, a second solvent consisting essentially of a 30:70 mixture of water and ethanol (30% water and 70% ethanol by volume) was contacted with the first fraction as described above in Example 1 under continuous agitation at a temperature of about 25° C. to 45° C. for 2 to 6 hours. The liquid obtained from this contacting process was distilled under reduced pressure to remove the ethanol. The resulting fraction optionally may be spray dried, which in this example was implemented as described above to yield a solid with approximately 5 to 10% water content. This fraction thus obtained was designated as the “second fraction” or “Fraction B.” It constituted a yield of about 10-15% of pigments, sterols, cell-wall fibers, and mono-sulfated saccharides.

The third extraction stage comprises contacting the second fraction with a third solvent. The third solvent preferably comprises about 40 to about 70 percent, and more preferably about 60 percent, by volume of water, and about 30 to about 60 percent, more preferably about 40 percent, by volume of low molecular weight alcohol, preferably ethanol. In this third stage, the third solvent preferably is modified to have a pH of about 2.5, e.g., by titration with an appropriate acid. Depending upon the specific application, examples of suitable acids would include hydrochloric acid, sulfuric acid, citric acid, and the like. The third extraction stage preferably comprises contacting the second fraction with the third solvent for about 2 to about 12 hours, and more preferably about 4 to about 6 hours, while agitating continuously. The third solvent preferably is at temperatures and temperature ranges as described above for the first and second extraction stages, e.g., about 45° C. to 75° C. The third extraction stage yields a liquid from which the ethanol is removed by known separation techniques such as distillation. The fraction that remains, or the portion of it that remains after water removal, is referred to herein as the “third fraction” or “Fraction C.”

### EXAMPLE 3

As an illustration of this third stage extraction, a third solvent comprising 60% water and 40% ethanol by volume was prepared. Hydrochloric acid (2 molar) was used to adjust the pH of the water-ethanol mixture from an initial pH of about 7.0 to a pH of about 2.5 using known titration methods. The second fraction was contacted with this acidified third solvent for approximately 6 hours at about 45° C. to 75° C. with continuous agitation. The resulting liquid was distilled under reduced pressure to remove the ethanol and yield a liquid referred to herein as the “third fraction” or “Fraction C.” This fraction also optionally may be spray dried, which in this illustrative example was done as described above. It yielded substantially solid phase material or compound comprising about 10% of mono-, di-, tetra- and octa-sulfated polysaccharides, fluorotannins and fluoroglucinols.

The third solvent or Fraction C as more broadly described herein comprises, and more preferably consists of or consists essentially of, the compound referred to above for reducing myostatin concentration. It comprises, depending on the starting materials, the processing parameters, and other variables as described herein, various sulfopolysaccharides. These may include mono-, di-, tetra- and octa-sulfosaccharides. The third fraction produced by the method as set forth herein above, including in its broader aspects, comprises another aspect of the invention. Similarly, the method for reducing myostatin concentration of a subject comprising administering to the subject at least one sulfated polysaccharide in an amount effective to reduce the myostatin concentration of the subject as described above may be carried out using this third fraction as a source for the compound comprising the sulfated polysaccharides.

Testing has been performed to evaluate the effectiveness of compounds and methods according to the various aspects of the invention as to their affinity to the myostatin protein. These tests will now be described.

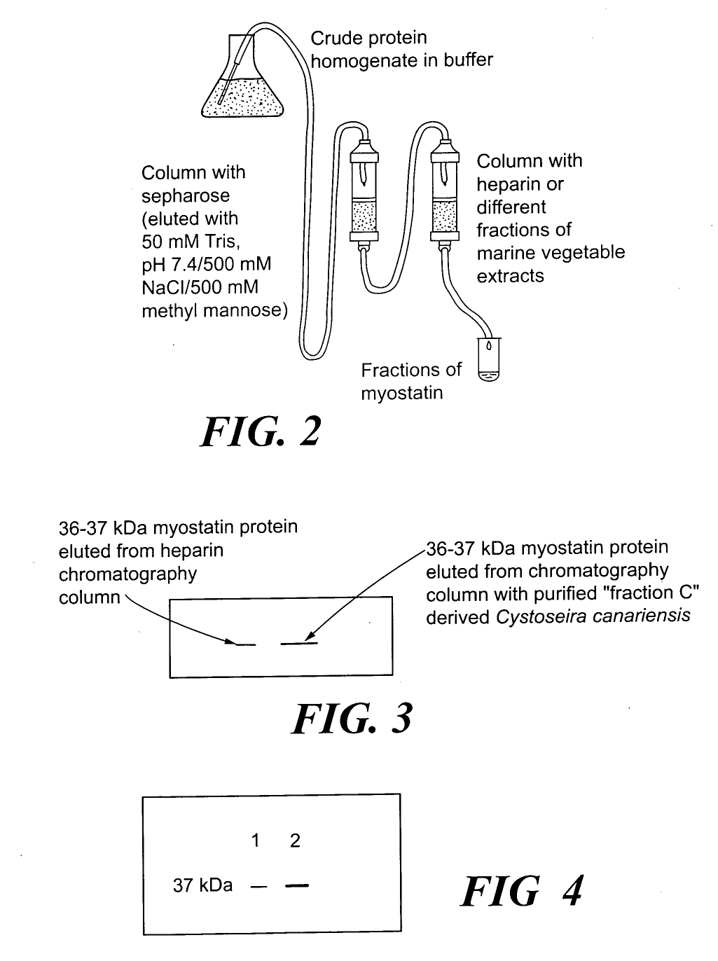

The testing involved the use of column chromatography analysis of Cystoseira canariensis fractions made as described herein. In the initial testing, the method described by Se-Jin-Lee and Alexandra McPherron, “Regulation of Myostatin Activity and Muscle Growth,” Proc. Nat'l Acad. Sci., Vol. 98, No. 16, pp. 9306-9311 (2001) (FIG. 1), was followed. FIG. 2 hereof provides an illustration of the experimental apparatus used. Crude muscle homogenate 50 extracted using a 200 M phosphate buffer (pH 7.2) from a specimen kindly provided by Las Palmas Hospital, Spain, after centrifuging, was passed over sepharose 52 (eluted with 50 mM Tris, pH 7.4/500 mM NaCl/500 mM methyl mannose) and heparin sepharose 54 (eluted with 50 mM Tris, pH 7.4/200 mM NaCl). Following this protocol it was possible to isolate the fraction containing the myostatin protein, revealed after Western-blot immunoelectro-phoresis of heparin sepharose elute.

Because of the apparent affinity of myostatin to heparin, the heparin chromatography column of the chromatography columns was replaced with various fractions derived from Cystoseira canariensis cultivated as described above.

To purify the fraction of proteins containing myostatin, we replaced the heparin chromatography column with a column filled with sulfated polysaccharides derived from Cystoseira canariensis as described herein. As a control, we used standard myostatin protein purification procedures described previously by Dr. Lee et al. 2001, noted above. Various fractions of eluate were collected, concentrated after acetone precipitation, and used.

Protein sodium dodecyl sulfate-polyacrylamide gel electrophoresis (“SDS-PAGE”) was performed with gradient gel from 10 to 20% acrylamide concentrations described by Laemmli UK, “Cleavage of Structural Proteins During 15 Assembly of the Head of Bacteriophage T4,” Nature, 227: 680-685 (1970). Western blot protein immuno-electrophoresis was performed according to the procedure previously described by Wehling M, Cai B, Tidball J., “Modulation of Myostatin Expression During Modified Muscle Use,” The FASEB Journal. 2000; 14:103-110. Proteins eluted from different fractions were prepared in SDS-PAGE reducing buffer (80 mM Tris-HCl pH 6.8, 0.1 M dithiothreitol, 70 mM SDS, 1.0 mM glycerol). Samples were boiled for 1 minute (min.), then centrifuged at 12,000×g for 1 min. The supernatant fraction of each sample was removed and used to determine protein concentration by measuring absorbance at 280 nanometers (nm). Homogenates containing 100 micrograms (μg) of total protein were separated on 10-20% lineal gradient SDS-PAGE gels according to Laemmli (1970), cited herein above. Proteins were electrophoretically transferred onto nitrocellulose membranes while immersed in a transfer buffer (39 mM glycine, 48 mM Tris).

After transfer, membranes were blocked in buffer containing 0.5% Tween-20, 0.2% gelatin, and 3.0% dry milk (blocking buffer) for at least 1 hour at room temperature. Membranes were probed with polyclonal anti-myostatin for 2 hours at room temperature. Subsequently, the membranes were overlain with alkaline phosphatase-conjugated anti-rabbit IgG for 1 hour at room temperature. After each incubation, the membranes were washed six times for 10 minutes in wash buffer (0.5% Tween-20, 0.2% gelatin, and 0.3% dry milk). Blots were developed using nitroblue tetrazolium and bromo-chloro-indolyl phosphate. The relative concentration of myostatin protein in each sample was determined by scanning densitometry.

Anti-Human Myostatin Antibody was raised in bovine against Human Myostatin his-Tagged Fusion Protein (BioVendor Laboratory Medicine, Inc., Palackeho tr. 56 612 00 Brno Czech Republic.Cat. No.: RD181005220). The recombinant human myostatin is 100% homologous with the human serum myostatin.

The sulfated polysaccharide fractions or components were characterized as follows. The molecular weights of the different fractions of fucan were determined by high-performance exclusion chromatograph (HPSEC), in 0.15 M NaCl, 0.05 M NaH₂ PO₄, pH 7, using a LICROSPHER.RTM.Si300 column (Merk-Clevenot) and a HEMASEC.RTM.BIO40 column (ALLTECH). The columns were calibrated with the following polysaccharide standards: pullulans: 853,000-5800 g/mol (Polymer Laboratories, Interchim), dextran: 1500 g/mol and milestone 522 g/mol (FLUKA), sucrose: 342 g/mol and glucose: 180 g/mol (SIGMA). The fucose content was determined as described by A. Dische, Method Biochem. Anal. 2, pp. 313-358, (1955). The sulfate content of the fractions was determined by elemental analysis of sulfur (S %), and by applying the following relation: percentage of sulfate groups (%)=3.22 times S %.

The following results were obtained. SDS-PAGE protein electrophoresis and Western Blot immuno-analysis revealed that antibodies raised against pure myostatin pro-peptide cross-recognized 36-37 kDa protein in both protein fractions eluted from heparin and sepharose and “Fraction C” plus sepharose chromatography column. The identity of purified protein as myostatin was confirmed after Western blot immuno-electrophoresis using antibodies raised against myostatin protein specifically, as is illustrated in FIG. 3. The first and second fractions (Fractions A and B) derived from Cystoseira canariensis as described above (Examples 1 and 2, respectively) did not show the same levels of affinity properties to the myostatin peptide.

Following this, muscle derived soluble protein was incubated with the third fraction (Fraction C) derived from Example 3 for 2 hours at 150° C. and then centrifuged. Western blot immunoelectrophoresis of the precipitate (pellet) and supernatant probed with antibodies raised against myostatin protein revealed cross-reaction with 36-37 kDa of protein in the precipitate (pellet), but not in the supernatant. Although not wishing to be bound by any particular theory, this could indicate that Fraction C cross-reacts with myostatin protein, which are precipitates during centrifugation. FIG. 4 shows a plot of the results. It shows a Western blot immunoelectrophoresis of proteins, in which Lane 1 shows the precipitate and Lane 2 shows the supernatant.

Thus, myostatin protein can be successfully isolated from muscle tissues using heparin-based chromatography as well as using sulfated polysaccharides extracted from cultured Cystoseira canariensis as described herein and designated as the third fraction or “Fraction C.” An analysis of the composition of “Fraction C” from the specific example described above revealed that it comprises 85% to 90% sulfated polysaccharides and fluorotannins, apparently specific to this plant. Although not wishing to be limited to any particular theory, it is possible that the fluorotannins might contribute some affinity of myostatin to the Fraction C chromatography column.

Laboratory studies have revealed that sulfated polysaccharide fractions isolated and purified from Cystoseira canariensis, e.g., Fraction C, possess specific binding effect to human myostatin protein in vitro. If previous laboratory observations were not coincidental artifacts and because myostatin acts as negative regulator of muscle cells in vivo, it was logical to assume that the administration of sulfated polysaccharides should promote muscle protein synthesis. To examine this hypothesis and to evaluate the pharmacological and physiological properties of sulfated polysaccharides, we studied the effect of sulfated polysaccharides and whey protein supplement of human muscle protein synthesis. We compared the rate of muscle protein synthesis in professional wrestlers after administration of 40 grams of whey protein concentrate per dose containing 500 mg of sulfated polysaccharides during a 60-day placebo-controlled clinical study. The study was performed at the Caucasian Olympic Center for Wrestling Training in Russia. The subjects volunteered, they were informed orally and in writing, informed consent was obtained, and the Ethics Committee of the Olympic Wrestling Center, approved the study.

The subjects included 18 (n=18) competitive professional wrestlers, aged 18 through 29. The subjects' body mass index (kg/m²) was between 22 and 25 kg/m². They were randomly divided into two groups. Thirty days before beginning the clinical study, the subjects underwent a period of diet counseling and surveillance. Their dietary intakes were standardized to contain 40 to 45% of total calories from carbohydrates. Subjects in the study were asked to maintain their professional training program throughout the trial.

The subjects were required to consume either 40 grams of whey protein supplemented with 500 mg of sulfated polysaccharide (Group 1, n=9) or 40 grams of whey protein (Group 2, n=9) used as placebo control. They took the approximately 40 grams of the material with 8 ounces of water, stirred until well blended, two servings daily.

On admission and after 60 days of the study, samples of muscle tissue were taken for evaluation of protein synthesis in muscle and mitochondria. Food record analysis, body mass index (BMI, kg/m²), and a symptom questionnaire were also included at the laboratory intervention times. Results are presented in Table 1, below.

TABLE 1
Results of Clinical Study.

| | | Body Mass | Fat Mass, | Fat-Free |
|---|---|---|---|---|
| Subjects | Age, yr | Index, kg/m² | kg | Mass, kg |
| Placebo | 21.3 ± 2.7 | 25.5 ± 2.1 | 17.2 ± 1.5 | 61.8 ± 3.5 |
| (n = 9 | | | | |
| SPs group | 21.7 ± 2.5 | 24.8 ± 1.7 | 18.1 ± 1.3 | 62.9 ± 3.8 |
| (n = 9) | | | | |

Values are means ± SE. Significant difference from control groups P < 0.05.

We measured rate of muscle proteins and mitochondrial proteins in serial muscle biopsy samples during a continuous infusion of L[1-¹³C] leucine from 8 subjects from the sulfa polysaccharides plus whey group (Group 1), and 7 subjects from the whey protein group (Group 2) under identical experimental conditions.

Muscle biopsies were taken with the needle biopsy technique from the lateral aspect of the quadriceps femoris muscle of both legs as described by Adey D, Kumar R, McCarthy J, Nair K S, “Reduced Synthesis of Muscle Proteins in Chronic Renal Failure,” J. Endocrinology and Metabolism, 278: 2; E219-E225 (2000). The biopsies were taken at a depth of 2-3 cm at about one-third of the distance from the upper margin of the patella to the anterior superior iliac spine. After local skin anesthesia, incisions through the skin and the muscle fascia were made (one on each leg) while subjects rested in the supine position. Biopsies from both legs were combined.

L[1-¹³C]leucine labeling was conducted as follows. L[1-¹³C]leucine (99 atom percent excess) were purchased from Cambridge Isotopes Laboratories (Andover, Mass.). Various isotopically labeled leucine solutions were prepared in sterile normal saline as described previously by Adey et al. (2000).

Mixed muscle proteins in the biopsy samples were separated and hydrolyzed as previously described by Baumann, P. Q., W. S. Stirewalt, B. D. O'Rourke, D. Howard, and K. S. Nair, “Precursor Pools of Protein Synthesis: A Stable Isotope Study In A Swine Model,” Am. J. Physiol. Endocrinol. Metab. 267: E203-E209 (1994). The isotopic enrichment of leucine in the mitochondrial hydrolysate also was measured with the same instrument, by use of a combustion system as previously described previously in Balagopal, P., G. C. Ford, D. B. Ebenstein, D. A. Nadeau, and K. S. Nair, “Mass Spectrometric Methods For Determination of [13C]Leucine Enrichment in Human Muscle Protein,” Anal. Biochem. 239: 77-85, (1996). Protein synthesis rate of a muscle protein was determined as previously described by Ljungqvist, O., M. Persson, G. C. Ford, and K. S. Nair, “Functional Heterogeneity of Leucine Pools in Human Skeletal Muscle,” Am. J. Physiol. Endocrinol. Metab. 273: E564-E570 (1997). Body weight was measured with a standard beam scale and the percentage of body fat from body density. The significance of differences between experimental and control samples was determined using one-way analysis of variance, with the confidence limit set at P<0.05. All values are given as means±SE.

Turning to the results, our clinical study demonstrated that under identical study conditions, the combination of sulfated polysaccharides and whey protein combination stimulates muscle and mitochondrial protein synthesis. The combination of whey protein with sulfated polysaccharide supplement produced far greater muscle protein synthesis by a mean (±SEM) of 47.1±1.4% than the placebo. The results more specifically revealed that, in subjects taking 40 grams in each of two servings per day of whey protein together with sulfated polysaccharides, both muscle and mitochondrial protein synthetic rates were significantly higher compared with subjects who consumed comparable amounts of whey protein alone. These results appear to indicate that sulfated polysaccharides possess significant pharmacological effect on muscle and mitochondrial protein synthesis. These results indicate that natural sulfated polysacharides are pharmacologically and physiologically very active compounds. The available literature indicates that sulfated polysaccharides in some instances can possess various pharmacological effects at very low concentrations. For example, it was previously demonstrated that sulfated polysaccharides bind to the angiogenesis inhibitor endostatin. Sulfated polysaccharides possess high affinity to fibroblast growth factor receptors. Endo-heparin sulfates isolated from several organs have been shown to interact with fibroblast growth factor receptors. These studies provide evidence that the interaction between fibroblast growth factor receptors and sulfated polysaccharides requires N-, 2-O and 6-O-sulfate groups.

The compounds and sulfated polysaccharide materials according to the various aspects of the invention may be used in various forms, such as solid, solid suspensions or slurries, solutions, etc. They may administered in various forms and using various administration methods. A preferred administration for the Fraction C as described herein and like compounds comprises encapsulating the material using known encapsulating techniques, or pressing them into tablet form, for oral administration. They may be taken alone, or in combination with other materials, such as other vitamin, mineral or supplement materials. Presently preferred dosages for oral administration are in the range of 500 mg to 1,000 mg daily, although this is not necessarily limiting.

Additional advantages and modifications will readily occur to those skilled in the art. For example, although preferred and illustrative sulfated polysaccharides and compounds according to various aspects of the invention have been described as extracts from Cystosiera canariensis, equivalent substances, e.g., such as structurally or functionally equivalent substances made synthetically or obtained from other sources, also may provide benefits as described herein. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

What is claimed is:

1. A compound for reducing myostatin concentration, the compound comprising an extract of Cystoseira canariensis comprising at least one sulfated polysaccharide.

2. A compound as recited in claim 1, wherein the at least one sulfated polysaccharide comprises a mono-sulfosaccharide.

3. A compound as recited in claim 1, wherein the at least one sulfated polysaccharide comprises a di-sulfosaccharide.

4. A compound as recited in claim 1, wherein the at least one sulfated polysaccharide comprises a tetra-sulfosaccharide.

5. A compound as recited in claim 1, wherein the at least one sulfated polysaccharide comprises an octa-sulfosaccharide.

6. A compound as recited in claim 1, wherein the at least one sulfated polysaccharide comprises a tetra-sulfosaccharide and an octa-sulfosaccharide.

7. A compound as recited in claim 1, wherein each of the at least one sulfated polysaccharides comprises fewer than nine saccharide units.

8. A compound as recited in claim 1, wherein the compound further comprises at least one fluorotannin.

9. A compound as recited in claim 1, wherein the compound further comprises at least one fluoroglucinol.

10. A method for reducing myostatin concentration, the method comprising applying to myostatin at least one sulfated polysaccharide.

11. A method for reducing myostatin concentration in a subject, the method comprising administering to the subject at least one sulfated polysaccharide in an amount effective to reduce the myostatin concentration in the subject.

12. A method as recited in claim 11, wherein the at least one sulfated polysaccharide comprises a mono-sulfosaccharide.

13. A compound as recited in claim 11, wherein the at least one sulfated polysaccharide comprises a di-sulfosaccharide.

14. A method as recited in claim 11, wherein the at least one sulfated polysaccharide comprises a tetra-sulfosaccharide.

15. A method as recited in claim 11, wherein the at least one sulfated polysaccharide comprises an octa-sulfosaccharide.

16. A method as recited in claim 11, wherein the at least one sulfated polysaccharide comprises a tetra-sulfosaccharide and an octa-sulfosaccharide.

17. A method as recited in claim 11, wherein each of the at least one sulfated polysaccharides comprises fewer than nine saccharide units.

18. A method as recited in claim 11, further comprising administering to the subject at least one fluorotannin.

19. A method as recited in claim 11, wherein the compound further comprises at least one fluoroglucinol.
